(19) United States
(12) Patent Application Publication
(10) Pub. No.: US 2025/0182870 A1
(43) Pub. Date: Jun. 5, 2025

(54) SYSTEM AND METHOD FOR MOBILE MEDICATION TRACKING

(71) Applicant: Log Rx Corporation, Sacramento, CA (US)

(72) Inventors: Clive Savacool, Sacramento, CA (US); Tanner Burton, Lehi, UT (US)

(73) Assignee: Log Rx Corporation, Sacramento, CA (US)

(21) Appl. No.: 18/528,512

(22) Filed: Dec. 4, 2023

(51) Int. Cl. G16H 20/10; G16H 10/40; G16H 15/00; G16H 40/20

(52) U.S. Cl. CPC G16H 20/10; G16H 10/40; G16H 15/00; G16H 40/20

(57) ABSTRACT

A mobile system for tracking and managing government-regulated medication and associated activities includes portable medication securing units with medication vials and vial boxes configured to store the medication vials and labeled with box identifiers. The system includes mobile computing devices with scanners for optical identification of the vial, the box identifiers, and biometric identification of a primary user or witness. The system includes a digital platform having cloud-connectivity utilizing an API. The platform includes a mobile application installed on mobile devices operable by the primary user or witness, and having identification, activity, tracking, inventory, and multiuser verification functions to process and display medication data associated with the scanned medication securing units, the primary user, and the witness, and to record the medication data locally and to the cloud. The platform also includes a multi-functional administrative portal that enables access and management of the medication data in real-time via the cloud.

### FIELD OF THE INVENTION

This disclosure relates to the field of substance tracking systems, especially those associated with medications.

### BACKGROUND

Administering and managing controlled substances such as narcotics while following government regulations can be a complex and challenging process. It entails adherence to numerous laws and regulations governing many activities in the field of medicine. The Drug Enforcement Administration (DEA) enforces Titles II and III of the Controlled Substances Act of 1970 (CSA), which require importers, exporters, manufacturers, distributors, dispensers, and healthcare practitioners that handle controlled substances, collectively known as registrants, to register with the DEA. The DEA's Office of Diversion Control is responsible for ensuring that all controlled substance transactions take place within the closed system of distribution established by Congress. Registrants that violate the CSA or its implementing regulations may be subject to DEA administrative enforcement actions or may face civil penalties or criminal prosecution by the U.S. Department of Justice. When controlled substance transactions fall outside the closed system of distribution, the activity constitutes diversion.

Diversion refers to the transfer of controlled substances from legitimate channels to illegitimate channels of distribution or use. This can occur at any point in the supply chain, from manufacturing to dispensing, and can involve a range of actors, including healthcare providers, patients, pharmacists, and drug dealers. Diversion can lead to a range of negative consequences, including addiction, overdose, and death. It can also contribute to the spread of infectious diseases, such as HIV and hepatitis C, through the sharing of needles and other injection equipment.

Considering the seriousness of the consequences, great care must always be taken in managing these substances, leading to more rigorous processes that require much more attention to detail. For healthcare practitioners, seemingly mundane tasks that would proceed without much difficulty in other fields, can become burdensome when dealing with powerful, even controversial medicines on a daily basis. Proper administration of controlled substances is thus a time-consuming and resource-intensive process. One issue involves the documentation required for controlled substance transactions. Healthcare providers must maintain accurate records of all controlled substance transactions, including prescriptions, dispensing, and destruction. They must also comply with a range of regulations and guidelines related to storage, security, and disposal of these substances. Failure to comply with these regulations can result in significant penalties, including fines, loss of license, and criminal prosecution.

Verification of controlled substance transactions can be a challenge. Healthcare providers must be vigilant for signs of diversion and abuse, which can be difficult to detect. They must also navigate complex regulations and guidelines to ensure that they are prescribing and dispensing controlled substances appropriately and safely. Historically, a number of the above regulated transactions, including witness verification, have been tracked with pen and paper, leaving a large margin for error and lessening accountability.

The misuse of and addiction to opioids, including prescription pain relievers, heroin, and synthetic opioids such as fentanyl, has led to a national crisis that affects not only public health, but also the social and economic welfare of the country. The need for access to controlled substances for legitimate medical purposes must be balanced with the need to prevent diversion and abuse. To address this challenge, healthcare practitioners must have access to more efficient and effective means of distributing, managing, and documenting controlled substances.

### SUMMARY

Described herein are devices, systems, and methods for tracking and managing government-regulated medication and associated activities.

An implementation relates a mobile system for tracking and managing government-regulated medication and associated activities is provided, the system having portable medication securing units comprising medication vials, each vial having a cap portion and body portion and labeled with vial identifiers, and vial boxes configured to store the medication vials and labeled with box identifiers. The system also includes at least two mobile computing devices, each device having scanners configured for optical identification of the vial and box identifiers, the scanners further configured for biometric identification of a primary user or a witness, the primary user operating a first mobile device, the witness operating a second mobile device. The system further includes a digital platform having cloud-connectivity and utilizing an Application Programming Interface (API), the platform further comprising a mobile application installed on the mobile devices and operable by the primary user or witness, the application having identification, activity, tracking, inventory, and multiuser verification functions, these functions configured to process and display medication data associated with the scanned medication securing units, the primary user, and the witness, the functions further configured to record said medication data locally and to the cloud. The platform also includes an administrative portal operable by an administrator and having dashboard, tracking, inventory, trend-tracking, and report-generating functions, the medication data accessible and manageable via the portal functions in real-time via the cloud. The system is such that the multiuser verification function is configured to biometrically identify the witness on the second mobile device, provide a scannable witness code on the second mobile device for the primary user to scan using the first mobile device so to verify the identity of the witness, and record verification information associated with the verified witness identification event as medication data, and wherein the medication data is integrated into the digital platform via the cloud following the verified witness identification event.

In one example, each vial identifier includes a unique alpha-numeric code for tracking, the code being visible in both data matrix barcode form for scanning and printed form for manual entry, wherein the witness code is provided for a limited period of time, and wherein the locally recorded medication data is automatically uploaded to the digital platform and accessible from the portal via the API when cloud access is available.

In another example, the alpha-numeric code is six digits, the data matrix barcode form being positioned on the cap portion while the printed form is positioned on the body portion, wherein the vial identifiers are color-coded, each vial identifier being configured to fit any medication vial with a minimum width of 0.5″, and wherein each box identifier is a self-locking, tamper-evident, one-piece component with multiple locking positions, the box identifier further including a unique alpha-numeric code for tracking, the code being visible in both data matrix barcode form for scanning and printed form for manual entry.

In another example, the mobile application's identification function provides medication data that includes identification information, the identification information further including a medication name, vial code, dose information, expiration date, box code, and lot number for each successfully scanned vial, wherein the identification function is further configured to automatically scan the vial identifier or box identifier via the optical scanners when either identifier lies within an optimal viewing region, and wherein the identification function includes a scan status indicator.

In another example, the activity function is configured to allow the primary user to transfer, administer, or waste the medication after each scan, said activities being recorded as medication data that includes an incident number for each activity, wherein the mobile application's tracking function uses GPS to track time-stamped locations of the vials or boxes when they are scanned, checked, transferred, administered, or wasted, said tracking information being recorded as medication data, wherein the mobile application further comprises a daily check function that requires a physical scan of the medication vial or box, wherein the multiuser verification function includes a user identification function that is configured to verify the identity of the primary user, alternate users, or guests via login credentials or other verification information, wherein the verification information further comprises date, time, and witness information associated with the verified witness identification event, and wherein the mobile application's inventory function is configured to display the medication data, the medication data further including quantity information associated with the medication securing units.

In another example, the administrative portal's inventory function further comprises a high level function and detailed function for displaying grouped and individualized medication data respectively, wherein the portal's tracking function uses GPS to track the time-stamped locations of the vials and boxes, wherein the portal further includes a searchable manifest function configured to display any individual vial included in the medication data, wherein the portal further includes a usage data function configured to monitor and manage the medication activity data, including administered vs. wasted statistics associated with the scanned vials, wherein the trend-tracking function is configured to track the time of every daily check and visually provide trends and outliers in the associated medication data, and wherein the report-generating function is configured to provide one-touch comprehensive report generation associated with the medication data.

Another implementation relates to a method utilizing a system for tracking and managing medication and associated activities, the method comprising Logging in a primary user to a digital platform's mobile application on a first mobile computing device; Navigating to a drug identification function found in the mobile application; Scanning a medication vial or vial box via a vial identifier or box identifier, respectively; Selecting an activity for the scanned item via an activity function found in the mobile application; Verifying the activity via a multiuser verification function found in the mobile application and using a witness by selecting biometric identification; Logging in the witness to the mobile application on a second mobile computing device; Providing biometric identification of the witness on the second mobile computing device; Generating a witness code on the second mobile computing device; Scanning the witness code on the first mobile computing device to complete verification; If the first mobile computing device is offline, then saving medication identification data, medication activity data, and witness verification data locally; If the first mobile computing device is online, then saving the above data to the digital platform via the cloud; Viewing the data on the first mobile computing device; and Logging in an administrator to the digital platform's administrative portal on a third computing device for monitoring and management of cumulative medication, user, and witness data.

In one example, the activities include administering, transferring, or wasting the scanned item.

In another example, verifying the activity includes an option for self-verifying the primary user, wherein the witness can be an alternate user or a guest, wherein if selecting self or alternate user, then completing verification following successful identification of the primary user or alternate user, the alternate user entering login credentials on the first mobile computing device, and wherein if selecting guest, then providing guest information on the first mobile computing device.

In another example, the data-saving to the cloud occurs automatically once the first mobile computing device gains cloud access.

In another example, the data-viewing on the first mobile computing device includes accessing tracking and inventory functions found in the mobile application.

In another example, the monitoring and management of the cumulative data on the administrative portal includes accessing dashboard, tracking, inventory, trend-tracking, and report-generating functions found in the portal.

Another implementation relates to a system for mobile substance tracking, the system comprising substance securing units, at least one mobile computing device, the device(s) having scanners configured for identification of the substance securing units, the scanners further configured for biometric identification of a primary user or a witness, and a digital platform having cloud-connectivity. The platform further comprising a mobile application installed on the mobile device(s) and operable by the primary user or witness, the application configured to process, display, and locally record substance data associated with the scanned substance securing units, the primary user, and the witness. The mobile application including a multiuser verification function configured to biometrically identify and verify the witness via the scanners, wherein the substance data is uploadable into the digital platform via the cloud. The platform also comprising an administrative portal operable by an administrator and configured to cumulatively monitor and manage the substance data recorded by the mobile application in real-time via the cloud.

In one example, the substance securing units comprise substance vials stored in vial boxes, the vials having vial identifiers, the boxes having box identifiers, both types of identifiers being scannable.

In another example, the multiuser verification function is configured to biometrically identify the witness on a second mobile device, provide a scannable witness code on the second mobile device for the primary user to scan using a first mobile device so to verify the identity of the witness, and record verification information associated with the verified witness identification event as substance data, and wherein the witness code is provided for a limited period of time.

In another example, the mobile application further includes identification, activity, tracking, and inventory functions for processing, displaying, and recording the substance data, and wherein the locally recorded substance data is automatically uploaded to the digital platform and accessible from the portal via an Application Programming Interface (API) when cloud access is available.

In another example, the administrative portal includes dashboard, tracking, inventory, trend-tracking, and report-generating functions for monitoring and managing the substance data.

In another example, the identification function provides substance data that includes identification information scanned from the vials and boxes, wherein the activity function is configured to allow the primary user to transfer, administer, or waste a substance after each scan, wherein the mobile application's tracking function uses GPS to track time-stamped locations of the vials or boxes when they are scanned, checked, transferred, administered, or wasted, wherein the mobile application's inventory function is configured to display the substance data, the substance data further including quantity information associated with the substance securing units, and wherein the multiuser verification function includes a user identification function that is configured to verify the identity of the primary user, alternate users, or guests via login credentials or other verification information.

In another example, the administrative portal further includes a manifest function configured to search for and display any individual substance vial in the inventory, and a usage data function configured to monitor and manage substance data associated with the actions taken with the activity function, wherein the administrative portal's inventory function further comprises a high level function and detailed function for displaying grouped and individualized substance data respectively, and wherein the portal's tracking function uses GPS to track the time-stamped locations of the vials and boxes.

In another example, each vial has a cap portion and body portion, wherein each vial identifier includes a unique alpha-numeric code for tracking, the code being visible in both data matrix barcode form for scanning and printed form for manual entry, the data matrix barcode form being positioned on the cap portion while the printed form is positioned on the body portion, and wherein each box identifier is a self-locking, tamper-evident, one-piece component with multiple locking positions, the box identifier further including a unique alpha-numeric code for tracking, the code being visible in both data matrix barcode form for scanning and printed form for manual entry.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

FIG. 1 illustrates a high-level diagram of a digital platform having cloud connectivity in a system for mobile medication tracking in accordance with an embodiment of the present disclosure.

FIG. 2 illustrates a flowchart outlining a series of steps taken by users operating a digital platform in a system for mobile medication tracking in accordance with an embodiment of the present disclosure.

FIGS. 3A-C illustrate a medication securing unit and its contents in accordance with an embodiment of the present disclosure.

FIG. 4 illustrates a screenshot of a home screen in a mobile application of a digital platform in accordance with an embodiment of the present disclosure.

FIG. 5 illustrates a screenshot of a biometric code identification function screen in a mobile application of a digital platform in accordance with an embodiment of the present disclosure.

FIG. 6 illustrates a screenshot of a medication identification function screen highlighting a scanned medication vial in a mobile application of a digital platform in accordance with an embodiment of the present disclosure.

FIG. 7 illustrates a screenshot of an activity function screen for a medication vial in a mobile application of a digital platform in accordance with an embodiment of the present disclosure.

FIG. 8 illustrates a screenshot of an activity function screen for administering medication in a mobile application of a digital platform in accordance with an embodiment of the present disclosure.

FIG. 9 illustrates a screenshot of a multiuser verification function screen for selecting a witness in a mobile application of a digital platform in accordance with an embodiment of the present disclosure.

FIGS. 10A-B illustrate screenshots of multiuser verification function screens for biometric identification in a mobile application of a digital platform in accordance with an embodiment of the present disclosure.

FIG. 11 illustrates a screenshot of a multiuser verification function screen for generating a code in a mobile application of a digital platform in accordance with an embodiment of the present disclosure.

FIG. 12 illustrates a screenshot of a tracking function screen in a mobile application of a digital platform in accordance with an embodiment of the present disclosure.

FIG. 13 illustrates a screenshot of a daily check function screen in a mobile application of a digital platform in accordance with an embodiment of the present disclosure.

FIG. 14 illustrates a screenshot of an administrator dashboard screen in an administrative portal of a digital platform in accordance with an embodiment of the present disclosure.

FIG. 15 illustrates a screenshot of a high level inventory function screen in an administrative portal of a digital platform in accordance with an embodiment of the present disclosure.

FIG. 16 illustrates a screenshot of a detailed inventory function screen in an administrative portal of a digital platform in accordance with an embodiment of the present disclosure.

FIG. 17 illustrates a screenshot of a report-generating function screen in an administrative portal of a digital platform in accordance with an embodiment of the present disclosure.

FIG. 18 illustrates a screenshot of an administered medication report screen in an administrative portal of a digital platform in accordance with an embodiment of the present disclosure.

FIG. 19 illustrates a screenshot of a drug manifest function screen in an administrative portal of a digital platform in accordance with an embodiment of the present disclosure.

FIG. 20 illustrates a screenshot of a medication tracking function screen in an administrative portal of a digital platform in accordance with an embodiment of the present disclosure.

### DETAILED DESCRIPTION

In the following discussion, numerous specific details are set forth to provide a thorough understanding of the disclosed subject matter. However, those skilled in the art will appreciate that the present disclosed subject matter may be practiced without such specific details. In other instances, well-known elements, processes or techniques have been briefly mentioned and not elaborated on in order not to obscure the disclosed subject matter in unnecessary detail and description. Moreover, specific details and the like may have been omitted inasmuch as such details are not deemed necessary to obtain a complete understanding of the disclosed subject matter, and are considered to be within the understanding of persons having ordinary skill in the relevant art.

The present invention is optimized for use in the medical field, including any industry where medications or drugs must be properly tracked and administered to patients in various settings. This includes, but is not limited to, departments and practitioners operating in fire, EMS, veterinary, law enforcement, wildlife, and air transport industries. The present invention eliminates the need for traditional pen-and-paper logs used for tracking drugs and associated activities. It further enhances and streamlines the accurate and careful tracking of drug administration activities both while working in the field and long after the activities occur, by digitizing and cloud-connecting the entire process. This includes the mobile tracking of full chain of custody operations, time-stamping of activities, tracking of individually encoded drugs and drug holders, drug inventory management, monitoring of field operators, and other related activities, with added administrative oversight and generation of cumulative analytics and reports over time. The result is much improved accountability for healthcare providers who are DEA registrants, and the mitigation of diversion outside of the medical field's closed system of distribution. The present invention is particularly useful for those practitioners operating in unpredictable emergency situations, where unknown external events can complicate inherently high-pressure and time-sensitive situations involving narcotics and other powerful controlled substances. Such practitioners can benefit from mobile medication tracking on the go. The term “medication” may be used interchangeably or in conjunction with others such as “drug”, “substance”, “controlled substance”, “pharmaceutical”, “narcotic”, “injection”, “Narcan”, “Naloxone”, “tranquilizer”, “sedative”, etc. Both multi-use and single-use drugs in a variety of measurements can be tracked, including their doses/dosages and expiration dates. A system and method are provided herein. The system is largely software-based, the software compatible with cloud-computing and used in conjunction with locked containers having a scannable exterior label, these containers securely storing smaller interior containers which hold substances and are further labeled for scanning. The words “information” and “data” may be used interchangeably, where “data” possesses a more digitized connotation, however a consistency of terms will generally be prioritized for integral features of the present invention, especially those claimed.

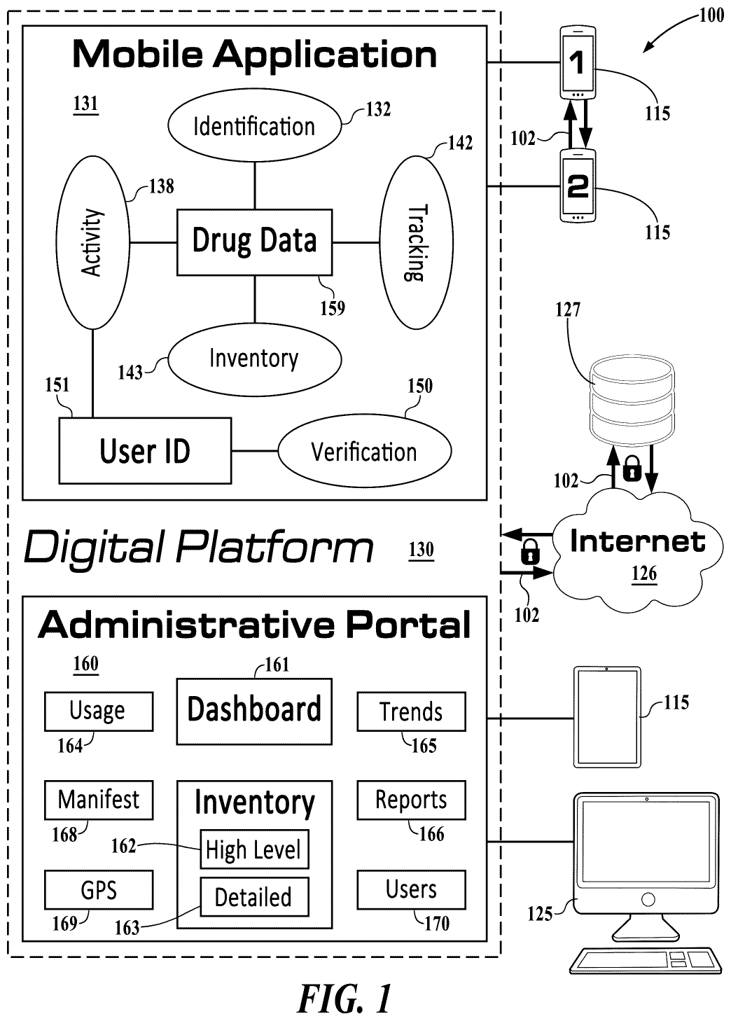

Referring to FIG. 1, an illustration shows a high-level diagram of a digital platform 130 having cloud connectivity in a mobile medication tracking system 100. An exemplary mobile system 100 allows for the local and remote tracking and management of government-regulated medication and activities associated with the medication. It comprises the digital platform 130 which further includes a mobile application 131 and an administrative portal 160. An exemplary digital platform 130 as a whole or via its constituent elements is securely connected to the Internet 126 or “cloud” for encrypted cloud-computing utilizing an Application Programming Interface (API), as indicated by two-way communication arrows 102. The associated network infrastructure is constructed or installed either via a secondary virtual database within the cloud, or by the secure transfer of data, again shown with arrows 102, to a primary physical database 127, or by some combination of both means.

The mobile application 131 can be installed on any number of mobile computing devices 115, including smartphones and tablets, while the administrative portal 160 can be accessed on any number of mobile computing devices 115 or desktop computing devices 125. The mobile devices 115 include standard scanners capable of optical identification of various items, including data matrix barcodes, these scanners or additional scanners further capable of biometric identification of a user, including facial recognition and/or fingerprint identification. The mobile application 131 is operable by a user; a primary user will be referred to in the present disclosure as the main user who initiates and completes the flow of mobile activities, including via a method, on a first mobile computing device 115, this device shown in the diagram with a number “1” displayed on its screen. A second mobile computing device 115 is shown with a number “2” displayed on its screen, and will, for purposes of discussing features of the present disclosure's system and method, be operable by a secondary user usually referred to as a “witness”, or verifier of drug activities carried out by the primary user on the first device 115. Other embodiments may envision an expanded plurality of computing devices, either mobile or desktop, operated in joint or shared capacities, including by other types of users such as alternate users and guests, to accomplish similar basic goals outlined herein, especially where legally compliant tracking and documentation of substances, their chain of custody, and associated activities are concerned.

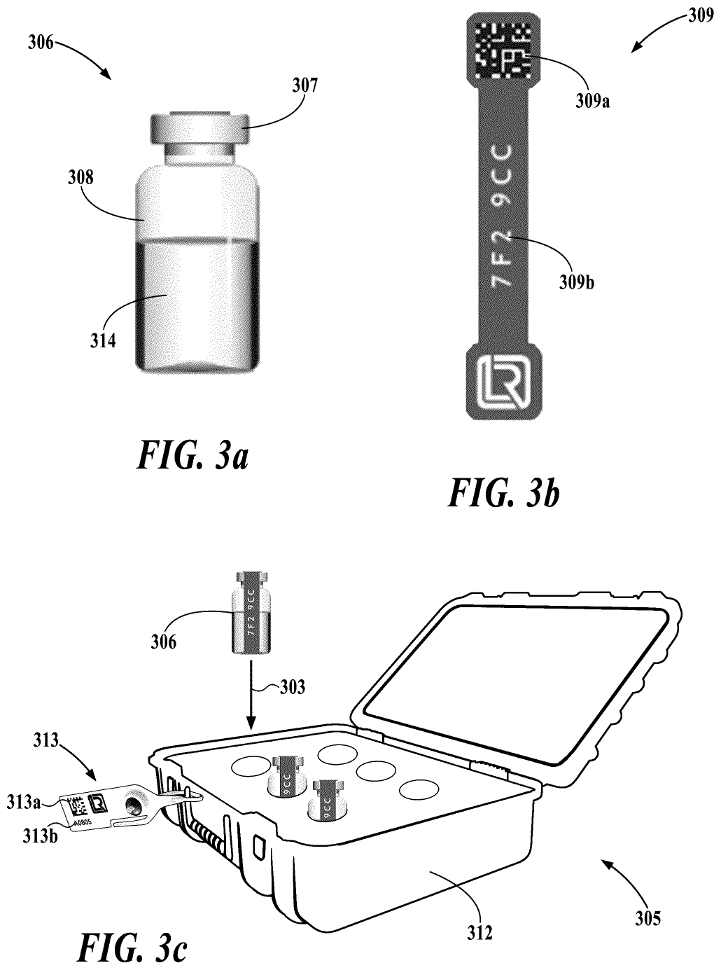

An exemplary mobile application 131 further comprises an identification function 132, a drug activity function 138, a drug tracking function 142, a drug inventory function 143, and a multiuser verification function 150, each function being associated with a screen or screens within the mobile application, the functions programmed to process and display substance/medication or drug data 159 associated with medication/substance securing units (see medication securing unit 305 of FIG. 3) having data matrix barcodes which have been scanned in via the identifying barcodes by the primary user on the first mobile computing device 115. The medication data 159 further and more broadly includes information associated with the primary user, and the witness, this information pertaining to their identification, actions taken while using the mobile application, and other user characteristics and activities. The above functions work within the mobile application 131 to record the medication data 159 locally on the first mobile device 115, and to the cloud 126, such that the uploaded medication data is integrated into the digital platform 130 via the API as a whole and available to a user of the platform, with proper access credentials where necessary—namely for the administrative portal 160.

In a series of steps, the multiuser verification function 150 allows the primary user to biometrically identify and verify the witness via the scanners found on the mobile device 115. An exemplary multiuser verification function 150 further comprises a user identification function 151 which includes a means for securely accepting login credentials such as a username and password from a user having an account registered with the digital platform 130. The user identification function 151 further comprises a biometrics function which can access the scanners found on the mobile device 115 to accept a user's fingerprint or utilize facial recognition to more quickly confirm the identity of a user. Using the biometrics function, the multiuser verification function 150 can biometrically identify the witness on the second mobile device 115, and respond to the identification by providing a scannable witness code on the second mobile device for the primary user to scan using the first mobile device in order to verify the identity of the witness. Two-way communication arrows 102 between the first and second mobile devices indicates the transfer of code data between them. Using this identification and verification procedure to complete a “verified witness identification event”, the primary user can successfully and accurately utilize a verifier for the various actions they take with medications using the activity function 138. With current government regulations, it may be most important for a primary user to complete verification for such drug actions or activities associated with the activity function 138, hence the connected elements-“Activity” 138, “User ID” 151, and “Verification” 150 shown in the diagram. However, considering changing laws, or varying standards and protocols, some healthcare practitioners or organizations/agencies may find it prudent to require witness verification every time a user utilizes any aspect of the application 131 to interact with substances. Thus, some embodiments may suggest witness verification after using other or all functions found in the application 131, including identification 132, tracking 142, and inventory 143. The mobile application 131 and/or multiuser verification function records verification information associated with the verified witness identification event as medication data 159, which is integrated into the digital platform 130 via the cloud 126 following the verified witness identification event. Some or all of the mobile application's functions may require witness verification before any local medication data 159 found on the mobile device 115 is allowed to be uploaded to the digital platform 130 as a cloud-connected whole, depending on government regulations and/or organization/agency protocol.

An exemplary administrative portal 160 offers a hub of medication data 159 where authorized administrators can securely login to monitor, manage, and learn from the cumulative data gathered in the field in order to maintain accurate records and further improve their organization's drug protocol in the future. The portal 160 is operable by an administrator and further comprises a dashboard function 161, a drug tracking function 169, a high level drug inventory function 162, a detailed drug inventory function 163, a trend-tracking function 165, a report-generating function 166, a drug usage data function 164, a drug manifest function 168, and a user monitoring function 170, the medication data 159 accessible and manageable via the portal functions in real-time via the cloud 126.

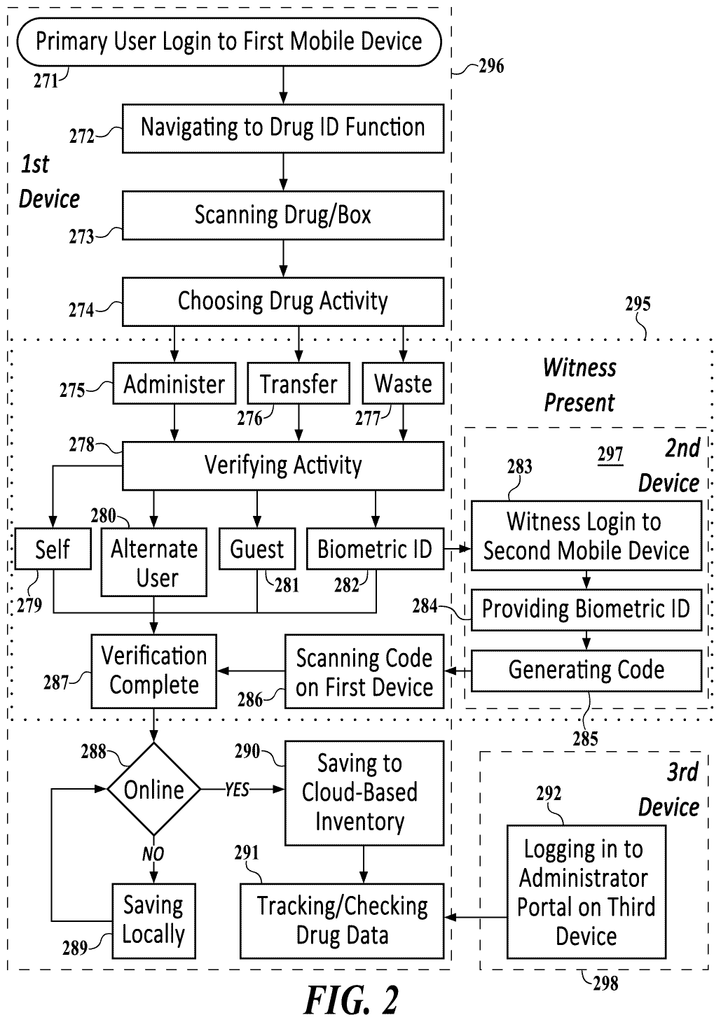

Referring to FIG. 2, an illustration shows a flowchart outlining a series of steps taken by users operating a digital platform in a system for mobile medication/substance tracking. These steps provide a method via which a primary user may verify actions taken with substances while using the activity function on the digital platform's mobile application installed on a first mobile computing device. Refer back to FIG. 1 or forward to subsequent figures showing screenshot images for various aspects of the digital platform, including mobile application and portal functions. Dashed box 296 includes steps taken on the first mobile device, dashed box 297 includes multiuser verification steps taken by a witness on a second mobile device, and dashed box 298 includes a step taken by an administrator utilizing the administrative portal on a third computing device. Dotted box 295 includes steps during which the witness should generally be present according to an exemplary embodiment and appropriate regulations.

Step 271 shows how the primary user begins by logging in to the digital platform's mobile application on a first mobile device. The application's user identification function (see “User ID” 151 of FIG. 1) can be used here either to accept login credentials or biometrics from the primary user. This initial login may access an aspect of or separate iteration of the user identification function that is not necessarily found within the multiuser verification function or on that particular screen. In step 272, the primary user navigates to the drug identification function found in the mobile application. Using this function, the primary user may utilize the mobile device's scanners to scan the labeled contents of medication securing units (see securing unit 305, vials 306, and box 312 of FIG. 3), including substance vials and vial boxes having scannable vial identifiers or box identifiers, respectively, as noted in step 273. At this point, the primary user enters a drug activity screen to select an action/activity for the scanned item via the activity function, as noted in step 274. Starting at steps 275-277, which indicate options to administer, transfer, or waste, respectively, the scanned item, the witness should ideally be present to monitor what is happening. At step 278, the primary user has taken one or more of the above actions, and navigates to the multiuser verification function screen in order to begin a verification procedure using the present witness. Alternatively, the primary user, typically in an emergency situation, may choose to self-verify the activity, as noted in step 279. As well, the primary user may select an alternate user as a witness, the alternate user either sharing an account with the primary user or having their own account on the digital platform, the alternate user then entering their own login credentials onto the first mobile device, as noted in step 280. Similarly, a guest may be logged as a witness in some manner, by providing guest information, as noted in step 281. The guest option may serve especially useful in emergency situations, when searching for a proper witness can prove very difficult and time is of the essence. If any of steps 279-281 are taken, then the primary user completes verification following successful identification of the self or witness via entering their respective login credentials on the first mobile device, as noted by step 287.

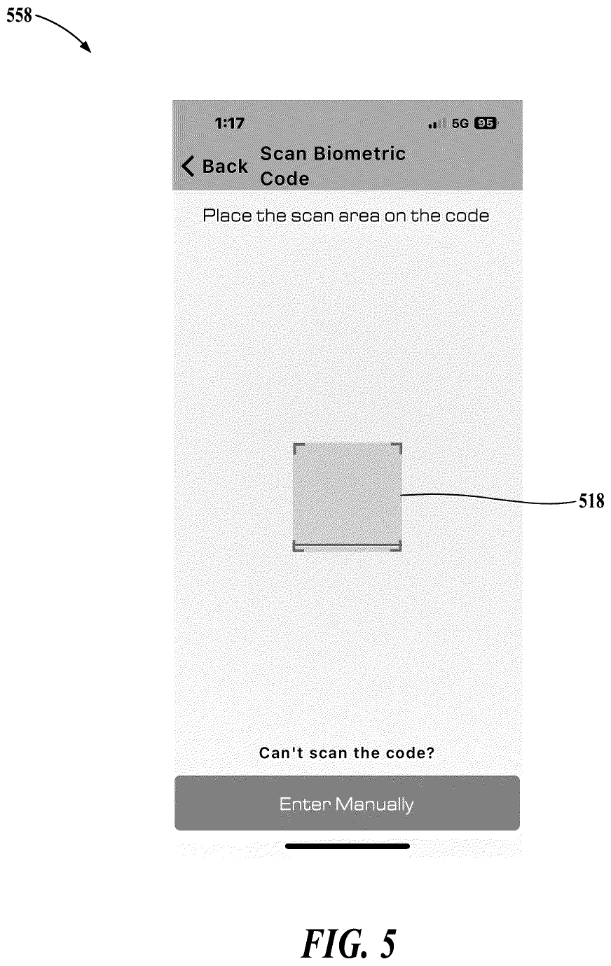

Step 282 indicates the option of biometrically identifying the witness via a second mobile computing device operated by the witness; an exemplary multiuser verification screen presents this option to the primary user as a biometrics icon such as a fingerprint. Once the primary user selects the biometric ID option 282, the witness should then login to the second mobile device, either using login credentials or biometrics, as noted in step 283. In step 284, the witness navigates to a biometrics feature, which an exemplary embodiment provides as a fingerprint icon found on the home screen of the application, and provides biometric identification on the second device. Other biometrics icons may be implemented in other examples, including those representing facial recognition. In response to successful biometric identification of the witness, a witness code is then generated on the second mobile computing device, as indicated by step 285. In step 286, the primary user then scans the witness code via a biometric code-scanning screen (refer to FIG. 5) in the application on the first mobile device. This marks the completion of successful witness verification via biometric identification, as noted by step 287.

Step 288 indicates a conditional step with an outcome dependent upon the availability of internet access from the first mobile device. Step 289 shows that if the first mobile computing device is offline from the internet, then the application automatically saves medication identification data, medication activity data, and witness verification data locally to the application and first device, this data derived from the above-mentioned usages of the respective mobile application functions. Step 290 shows that if the first mobile computing device is online, then the application automatically saves the above data to the digital platform via the cloud. In either circumstance, local saving may occur automatically regardless of network availability. Moreover, in an exemplary embodiment, data-saving to the cloud occurs automatically once the first mobile computing device gains cloud access. The primary user can then view the medication data from within the mobile application, as indicated by step 291. In some embodiments, the primary user can view and track the medication data on the first mobile computing device whether the device has cloud access or not, while in a preferred embodiment, cloud access may be required. Data-viewing on the first mobile computing device includes accessing tracking and inventory functions found in the mobile application. In step 292, an administrator logs in to an administrative portal of the digital platform on a third computing device in order to monitor and manage all cumulative medication, user, and witness data incoming from the field. In an exemplary embodiment, this cumulative data-viewing includes accessing dashboard, tracking, inventory, trend-tracking, and report-generating functions found in the portal. Other embodiments may further include a usage data function and/or user monitoring/data function via which other types of medication data may also be monitored.

Referring to FIGS. 3A-C, illustrations show a medication/substance securing unit 305 and its contents, the contents including medication vials 306 securely stored inside of a vial box 312. Each vial 306 further comprises a cap portion 307 and body portion 308, each vial 306 being labeled with a vial identifier 309, FIG. 3a showing an unlabeled vial 306, FIG. 3b showing the identifier 309 on its own, while FIG. 3c shows the identifiers 309 adhered to the vials 306. Where visible, the vials 306 are shown having some type of substance/medication 314 filling some of the body portion 308. An exemplary vial box 312 is also labeled or attached with a box identifier 313. An exemplary box identifier 313 is a self-locking, tamper-evident, one-piece component with multiple locking positions, the device shown in FIG. 3c hooked or latched around a handle for the box 312. Other embodiments envision other protrusions from the box 312 that can serve as the anchor point for the box identifier 313. In one example, the box identifier 313 is a dual-purpose device that can securely lock the box 312 when latched. In another example, the box identifier 313 is an adhesive label placed upon a surface of the box 312, and the box is separately locked by a dedicated locking mechanism joining the lid and container portion of the box when it is closed. The container portion of the box 312 can further be covered with a protective foam material to cushion the substance vials 306, the material having openings of an ideal depth and diameter to accept the vials, keeping them protected and stationary regardless of box movements and vibration. Insertion arrow 303 helps to illustrate the placing/securing of a vial 306 into the box 312. In a preferred embodiment, both the vial identifiers 309 and box identifiers 313 are scannable by standard optical identification scanners found on mobile computing devices.

Further in a preferred embodiment, each vial identifier 309 includes a unique six digit alpha-numeric vial code for tracking, the code being visible in both data matrix barcode form 309a for scanning, and printed form 309b for manual entry, as illustrated in FIG. 3b. With the vial identifier 309 applied to the medication vial 306, the data matrix barcode form 309a is positioned on a top surface of the cap portion 307, while the printed form 309b is positioned heightwise along the vial's body portion 308, such that the readable or scannable surfaces of the codes are positioned orthogonally relative to each other when the vial cap is installed onto the vial. In this way, a user can scan the top of each vial 306 while it sits within the foam in the vial box 312, without having to remove and replace each vial for every scan. In one example, for further enhanced organization, the vial identifiers 309 can be color-coded. Exemplary vial identifiers 309 can fit any medication vial 306 with a minimum width of 0.5″.

Similarly to the codes found on the vial identifiers 309, each exemplary box identifier 313 includes a unique alpha-numeric box code for tracking, the code being visible in both data matrix barcode form 313a for scanning, and printed form 313b for manual entry, as illustrated in FIG. 3c. The code forms can be printed on opposing sides of the box identifier 313, or on the same side as illustrated in the drawing.

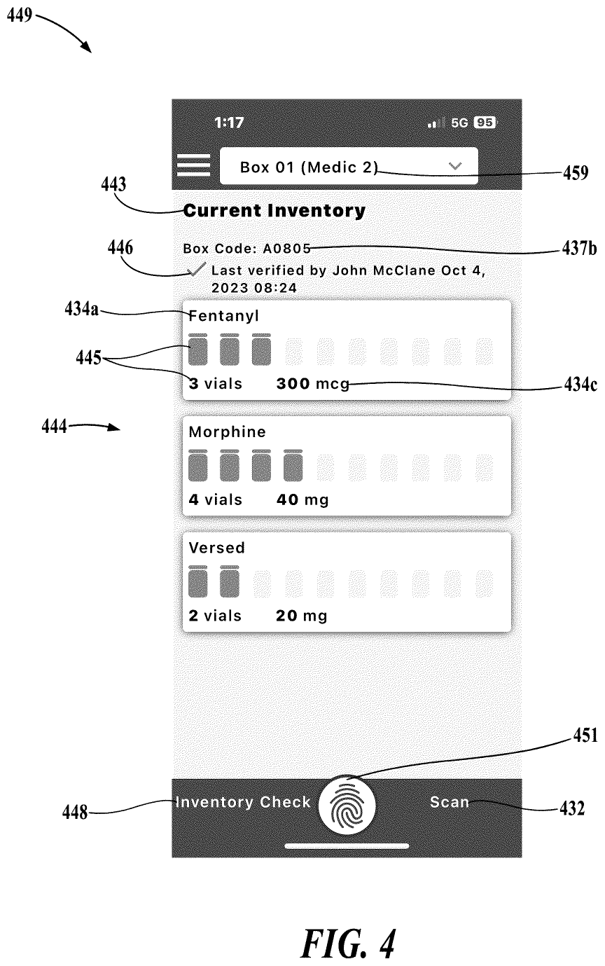

Referring to FIG. 4, an illustration shows a screenshot of a home screen 449 in the mobile application of the digital platform. In the embodiment illustrated, the home screen 449 displays information associated with the inventory function 443, or an inventory screen. The screen includes a pulldown menu with various selectable inventory items associated with aspects/forms of medication data 459, the currently selected data including a vial box number and medic number (“Medic 2” being responsible for “Box 01” in this example). Data grouped thusly may be seen in subsequent administrative portal figures as “unit” information related to any single resource capable of providing or administering a controlled substance, and generally associated with vial box location (e.g. via the tracking function), agency, and/or user data, and distinct from the aforementioned medication/substance securing units.

Medication data further comprises identification information 444. The current screen further provides the identification information 444 pulled from the application's identification function, including vial, box, and lot information such as identifying codes and/or numbers gathered from scanning, dose info, and expiration dates. The screen further shows verification information 446 documented from procedures completed using the application's multiuser verification function. Exemplary verification information comprises date, time, and witness information associated with the verified witness identification event. The screenshot also displays medication quantity information 445 as provided by the application's inventory function 443. On this screen, the box information includes an alpha-numeric box code 437b, or “A0805”. The identification information 444 further comprises medication names 434a, or “Fentanyl”, “Morphine”, and “Versed” specifically, as well as dose information 434c, which in one instance is 300 mcg of the Fentanyl. Other identification information can include an expiration date for medication stored in a scanned vial. In the same instance, the quantity information 445 indicates that there are “3 vials” remaining in the currently selected vial box. The verification information 446 includes a verifier or witness name (“John McClane”), along with the date and time at which this witness last verified this particular vial box, namely Box 01 having box code A0805.

The lower or bottom portion of the currently depicted screen includes links to other application functions, including a daily inventory check 448, the identification function 432, and the user identification function 451. This embodiment provides a link (the fingerprint icon) to the biometrics aspect of the user identification function 451, specifically the biometric identification of a witness seeking to verify a drug activity by using the application on the second mobile computing device.

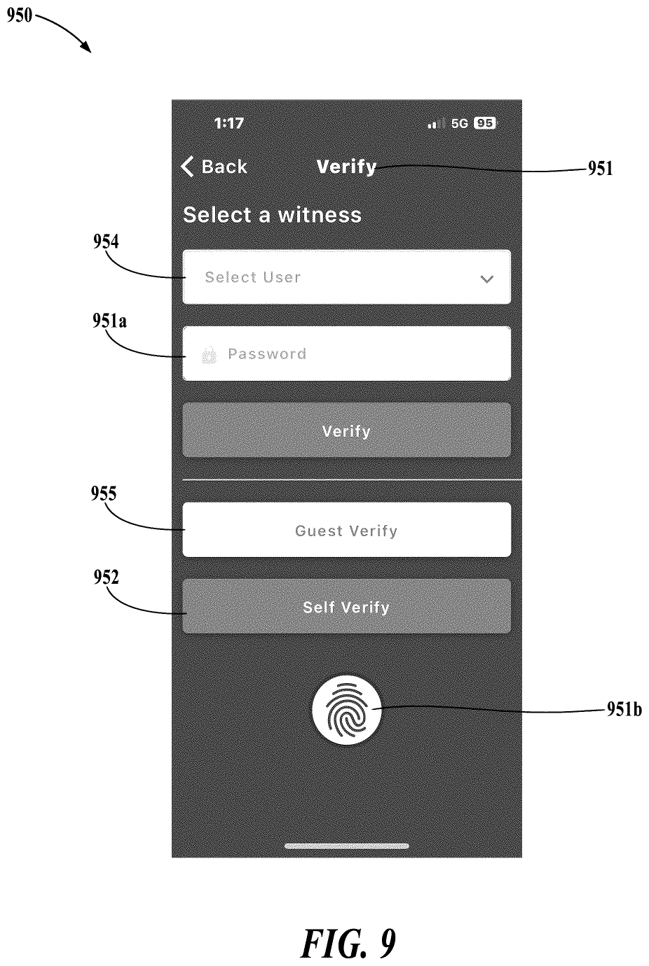

Referring to FIG. 5, an illustration shows a screenshot of a biometric code identification function 558 in a mobile application of a digital platform. The screen includes a standard scan area 518 via which a user can view a real-time feed from the aforementioned optical identification scanner (the rear-facing camera, in most cases). In a preferred embodiment, the currently depicted screen is displayed on the first mobile device of the primary user following their opting for biometric verification of a drug activity on the main multiuser verification screen (see witness selection screen of FIG. 9). Using this biometric code identification screen, a primary user can direct their first mobile device with scan area 518 over a witness code generated by the verifier's second mobile device in order to capture the code and process/complete verification of the drug activity.

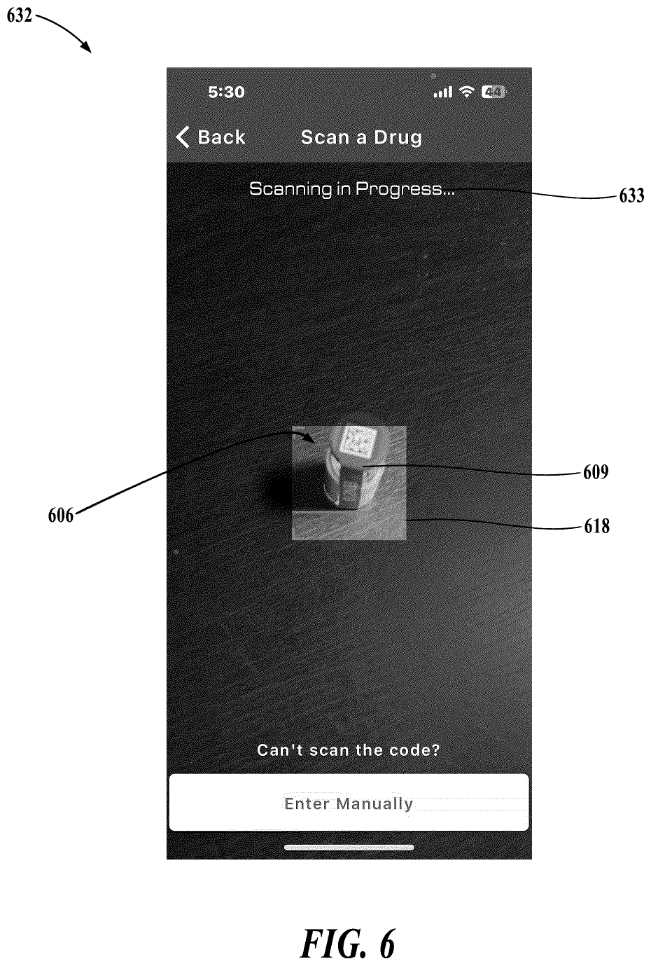

Referring to FIG. 6, an illustration shows a screenshot of a medication identification function 632 highlighting a scanned medication vial 606 in a mobile application of a digital platform. In this function of the application, the scan area 618 is directed over top of the vial 606 with vial identifier 609 in order to capture the identifier's data matrix barcode, or QR code. The identification function 632 can automatically scan the vial identifier 609 or box identifier via the optical scanners when either identifier lies within an optimal viewing region of the scan area 618. In a preferred embodiment, the identification function 632 includes a scan status indicator 633 which shows the progress of the vial scan, this embodiment using the text, “Scanning in Progress . . . ”. Another example of a scan status indicator 633 may utilize a loading bar or other related visualization along with text or a percentage, or on its own.

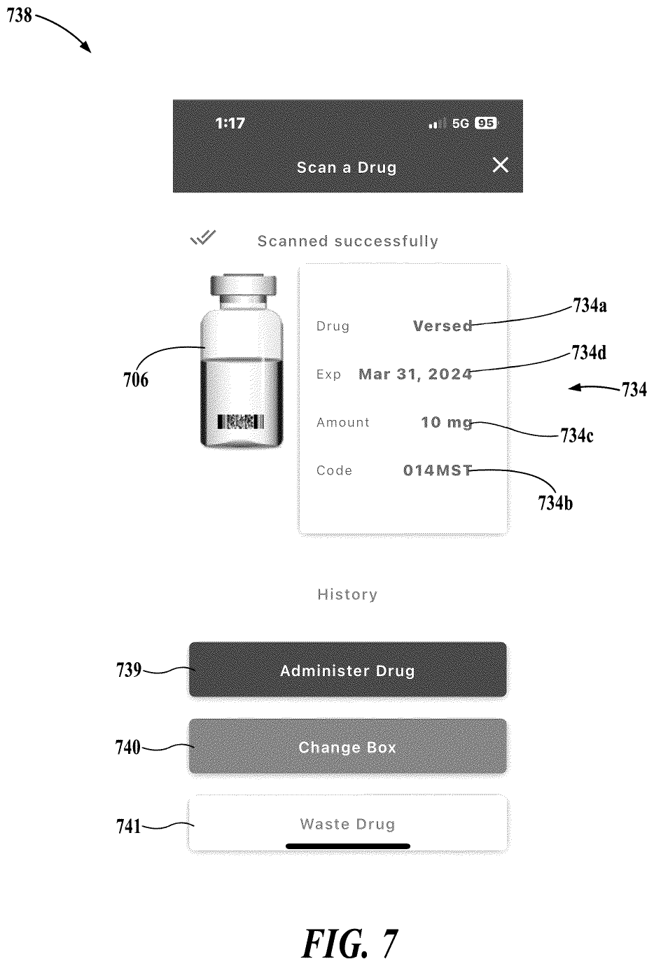

Referring to FIG. 7, an illustration shows a screenshot of an activity function 738 for a scanned medication vial 706 in a mobile application of a digital platform. The screen includes vial information 734 near the vial 706, the information further comprising the medication name 734a, its expiration date 734d, its dose information 734c, and its medication code 734b. Below the vial information 734 are several activities or actions the primary user can take with the displayed medication after it has been scanned. The activities include administering 739, transferring 740, and wasting 741 the drug. The user can select an activity by tapping it on the first mobile device. A verifier/witness is preferably present while the primary user performs the chosen activity. Medication data associated with the selected activity is recorded by the mobile application and digital platform as previously discussed. If the mobile device lacks or loses internet access, the locally recorded medication data is automatically uploaded to the digital platform when cloud access is available.

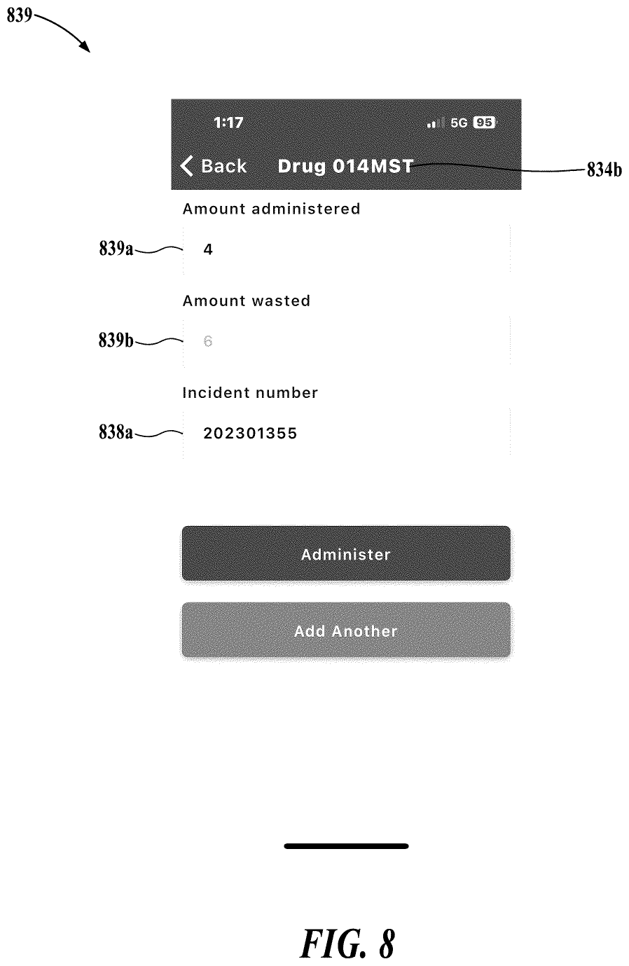

Referring to FIG. 8, an illustration shows a screenshot of the activity function's screen for medication administering 839 in a mobile application of a digital platform. The screen shows a medication code 834b prominently displayed near the top. The screen further displays other medication data associated with the administered drug, including an administered amount 839a and a wasted amount 839b. Finally, an activity incident number 838a is presented to help document and track the activity itself. In a preferred embodiment, such an incident number 838a can be associated with any activity or action performed by a user while using the activity function. In some examples, incident numbers 838a are associated with a plurality of or all application functions in order to facilitate real-time or subsequent tracking via the application and the administrative portal.

Referring to FIG. 9, an illustration shows a screenshot of a multiuser verification function 950 for selecting a witness, or witness selection screen, highlighted by a user identification function 951 (the “Verify” title referring to the user identification aspect of the multiuser verification function) in a mobile application of a digital platform. Using this screen, the primary user can verify their own identity (i.e. self-verification 952) or that of alternate users 954 or guests 955 via login credentials entered into a login or password field 951a, or via other verification information. An alternate user 954 of the mobile application or digital platform can be selected from the drop-down menu displaying the text “Select User”. Alternatively, the primary user can select a guest 955 by tapping the “Guest Verify” button or link. The guest option allows access to more healthcare practitioners on the scene who can serve as verifiers. Finally, the primary user can opt for the biometrics function 951b in order to begin the process for verification via biometric identification of a witness using a second mobile computing device.

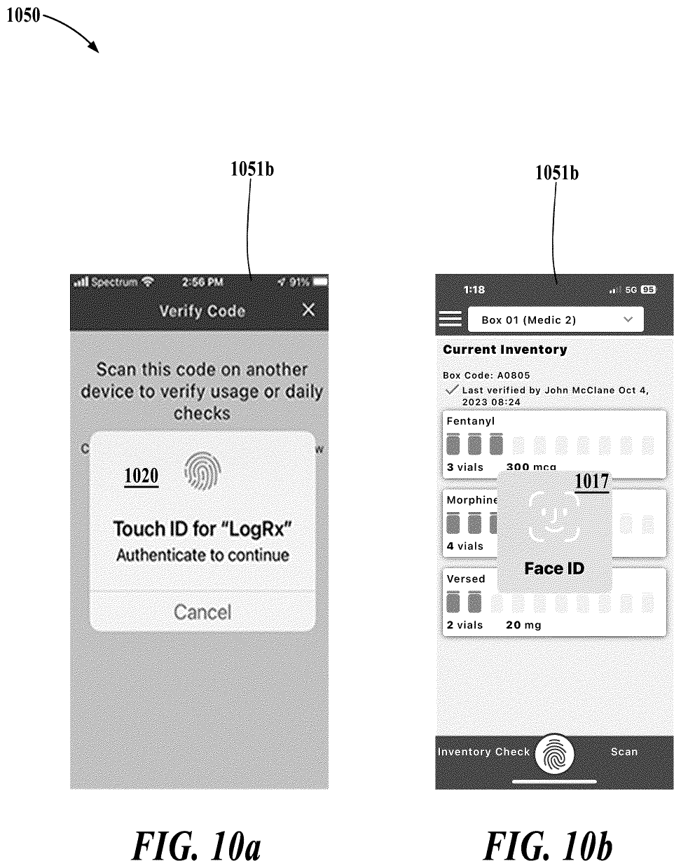

Referring to FIGS. 10A-B, illustrations show screenshots of multiuser verification function screens for biometric identification in a mobile application of a digital platform. Both screens present further aspects of the user identification function 1051b, although the type of biometrics available on a given mobile computing device can vary. The present invention aims to provide at least one type of biometric identification when using the mobile application. FIG. 10a depicts a screen for fingerprint scanning 1020, or “Touch ID” authentication. FIG. 10b depicts a screen for facial scanning 1017, usually with a front-facing camera of the mobile device. Using one of these identification options on the second mobile computing device, the witness can prove their identity during the verified witness identification event.

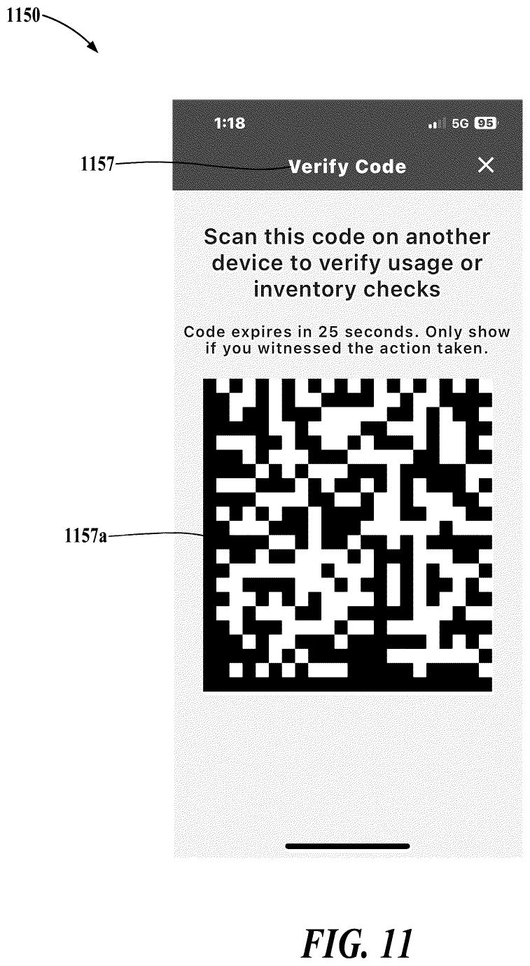

Referring to FIG. 11, an illustration shows a screenshot of an aspect of the multiuser verification function 1150 which generates a code in a mobile application of a digital platform. The “Verify Code” title indicates a witness-code-generating function 1157 within the multiuser verification function 1150. This function generates a large scannable witness code 1157a that is displayed on this screen of the second mobile device after the witness has biometrically self-identified on the device. In this embodiment, the code 1157a is in the form of a data matrix barcode that is provided for a limited period of time, or 30 seconds specifically in this example, with a countdown timer provided above the code. It is within this limited time frame that the primary user must scan this witness code 1157a using the first mobile device. Once the primary user performs this scan, the verified witness identification event is complete.

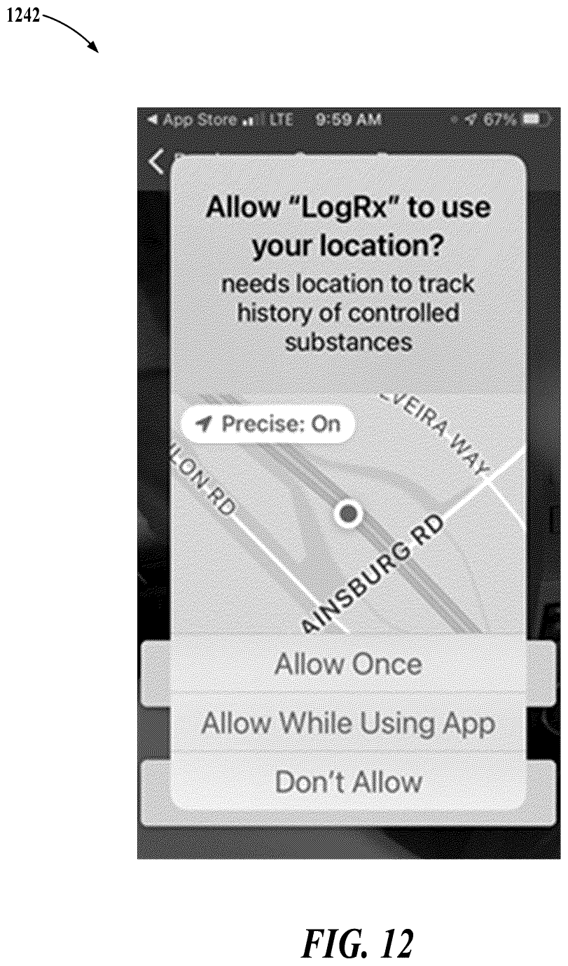

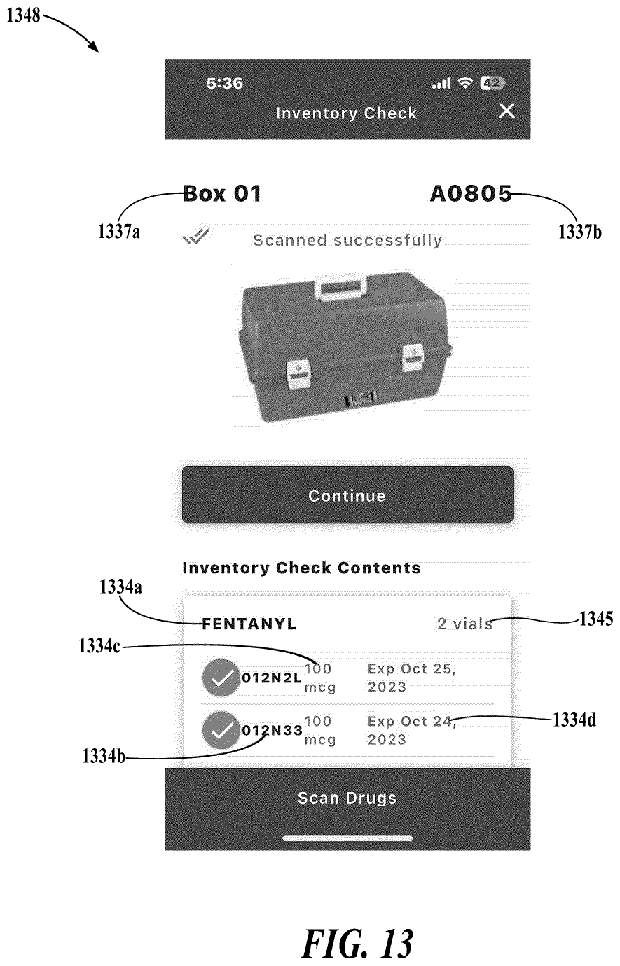

Referring to FIG. 12, an illustration shows a screenshot of a tracking function 1242 in a mobile application of a digital platform. An exemplary mobile tracking function 1242 uses GPS to track time-stamped locations of the substance vials or boxes from the time they are scanned and thereafter, including when they are checked (as accomplished with a daily inventory check function 1348, refer to FIG. 13), transferred, administered, wasted, or whenever taking any number of actions with any number of application functions. Tracking information associated with the above instances is recorded as medication data locally to the smartphone or tablet running the mobile application, and uploadable to the cloud-connected digital platform, including to the administrative portal.

Referring to FIG. 13, an illustration shows a screenshot of a daily check function 1348 in a mobile application of a digital platform. Various types of medication/substance data stored via the inventory function (in conjunction with the other app functions) can be displayed and viewed using the daily check 1348. The current screen provides a vial box number 1337a and vial box code 1337b scanned from the box identifier's data matrix barcode. The screen further provides quantity information 1345 for the vials stored in the box, along with vial information including the medication name 1334a, medication codes 1334b, dose information 1334c, and expiration dates 1334d. In a preferred embodiment, the daily check 1348 requires a physical scan of the checked medication vial or box, substantially enhancing accountability for organizations, agencies, and healthcare practitioners by promoting greater accuracy in record-keeping.

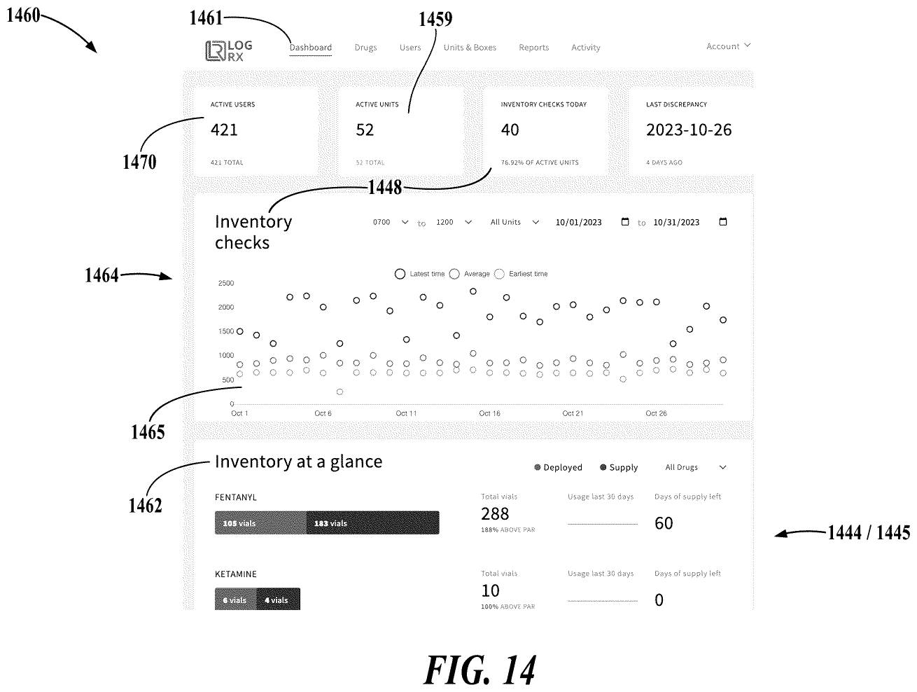

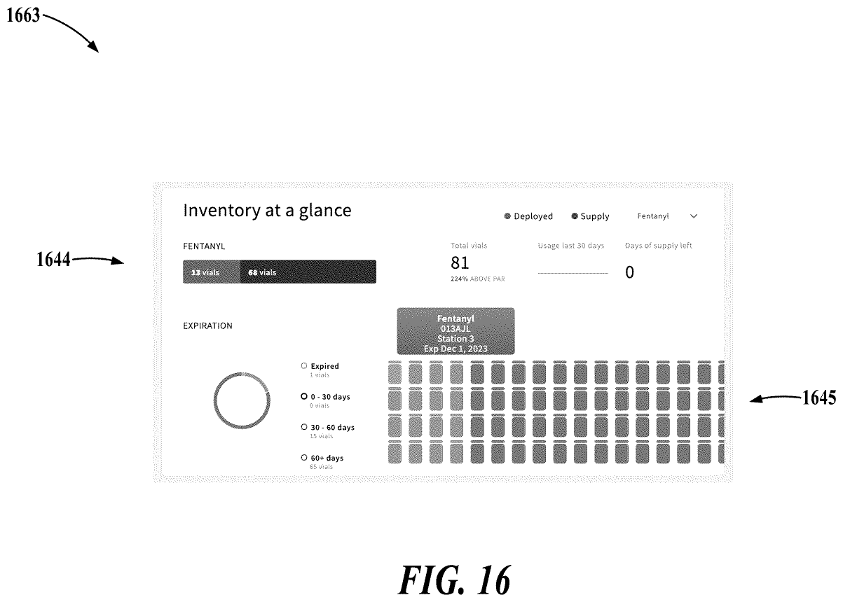

Referring to FIG. 14, an illustration shows a screenshot of an administrator dashboard 1461 in an administrative portal 1460 of a digital platform. In an exemplary embodiment, a number of portal functions simultaneously display their associated medication/substance data on the dashboard 1461 to provide an overview of the data. It should be noted that the portal 1460 can include features and functions that overlap with those of the mobile application in order to share and display medication data, but the portal may augment these features/functions to provide a more comprehensive view of the cumulative data, especially for the purpose of analysis. Like the mobile application, the portal 1460 includes an inventory function, an exemplary portal's inventory including a high level inventory function 1462 providing a broader view of vial/box identification information 1444 and quantity information 1445 that is viewed in groups, as well as a more narrowed, detailed inventory function for displaying individualized identification information (refer to the screenshot shown in FIG. 16 for detailed inventory function 1663).

The depicted embodiment of the dashboard function 1461 highlights medication data associated with the mobile application's daily check function 1448, including the current day's number of inventory checks (40 checks, specifically), and a percentage of active units (“units” here again referring to locationally derived or medic-related info, typically) associated with that number. The portal 1460 further includes usage data function 1464 which can monitor and manage medication data associated with the usage of app functions (e.g. the daily check and activity functions) and various activities or actions taken with vials/substances, including administered vs. wasted statistics associated with scanned medication vials. Using the usage data function 1464, the portal 1460 provides an overview of user behaviors (e.g. the frequency and types of daily checks) while operating the mobile application in the field. A trend-tracking function 1465 integrates the associated usage data into a form that can be analyzed by the administrator. The trend-tracking function 1465 can track the time of every daily check and visually provide trends and outliers in the associated usage data, or other medication data in other implementations of the function 1465. The present administrator dashboard 1461 displays medication data 1459 derived from one or more app functions (e.g. tracking) and generally associated with drug location data or user data (e.g. “52 active units”, again, a higher level categorization which is more agency-related and distinct from the drug securing units). The screen further displays medication data pulled from a user monitoring function 1470, which pertains more directly to the users themselves (e.g. “421 active users”, healthcare organization/agency statistics, etc).

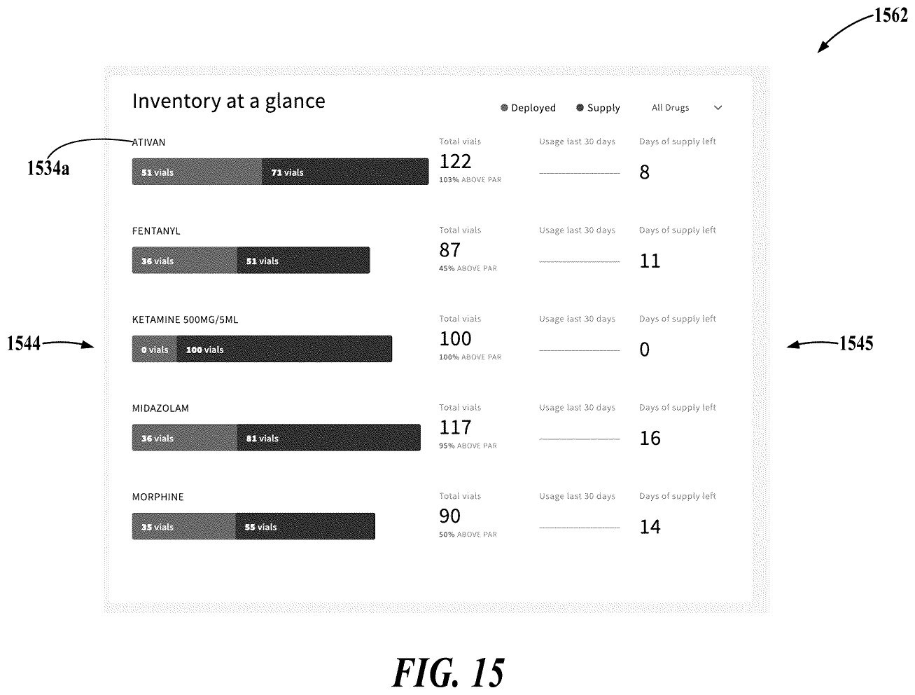

Referring to FIG. 15, an illustration shows a screenshot of a high level inventory function 1562 in an administrative portal of a digital platform. An exemplary screen for this portal inventory function 1562 includes identification information 1544, including vial information such as medication names 1534a (e.g. Ativan, Fentanyl, Ketamine, etc.). The screen further displays drug quantity information 1545, such as “Total vials” and “Days of supply left”, with associated quantity numbers. The high level inventory function 1562 groups blocks of medication data together for cumulative inventory analysis.

Referring to FIG. 16, an illustration shows a screenshot of a detailed inventory function 1663 in an administrative portal of a digital platform, the screen displaying more individualized medication identification information 1644 along with quantity information 1645. In this example, the Fentanyl inventory is highlighted, with a visual aid that conveys drug expiration data. Using this function screen, an administrator can target specific substances in the inventory for enhanced record-keeping and monitoring of associated medication data.

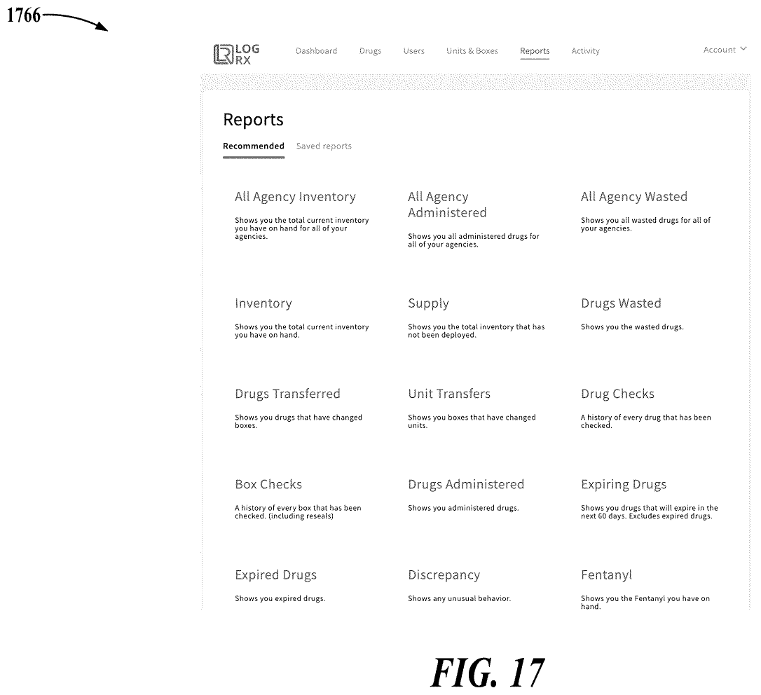

Referring to FIG. 17, an illustration shows a screenshot of a report-generating function 1766 in an administrative portal of a digital platform. This function 1766 can provide one-touch comprehensive report generation associated with many types of medication data. As shown, some examples of reports include: all agency or health organization reports (including for inventory, administered drug, and wasted drug data associated with the agencies), current inventory reports, supply reports, wasted drug reports, transferred vial reports, transferred box reports, drug check and box check reports (e.g. associated with daily checks or otherwise), administered drug reports, soon-to-expire drug reports, expired drug reports, discrepancy reports (e.g. for seeking outliers in usage and/or app user data), and individual drug reports (e.g. for Fentanyl or another narcotic). The administrator can login to the portal on a computing device (i.e. the aforementioned third device, either desktop or mobile-especially a tablet) to generate such reports and analyze them to formulate protocol that further improves accountability, thus substantially enhancing the management of medication data.

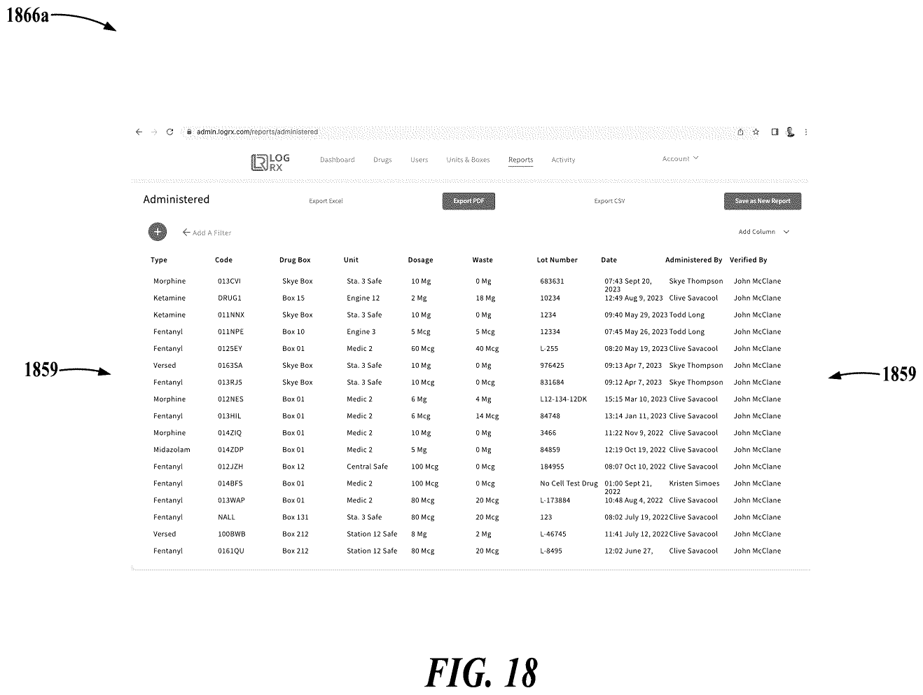

Referring to FIG. 18, an illustration shows a screenshot of an administered medication report 1866a in an administrative portal of a digital platform. The report 1866a includes such medication data 1859 as: drug names and codes, drug box names or numbers (some embodiments show scanned box codes), brief unit descriptions (e.g. a practitioner such as a medic, organization/agency shorthand, vehicle name, and/or location nickname, etc.), dosage info, wasted drug info, lot numbers, dates for the activity occurrence, practitioner names, and verifier/witness names.

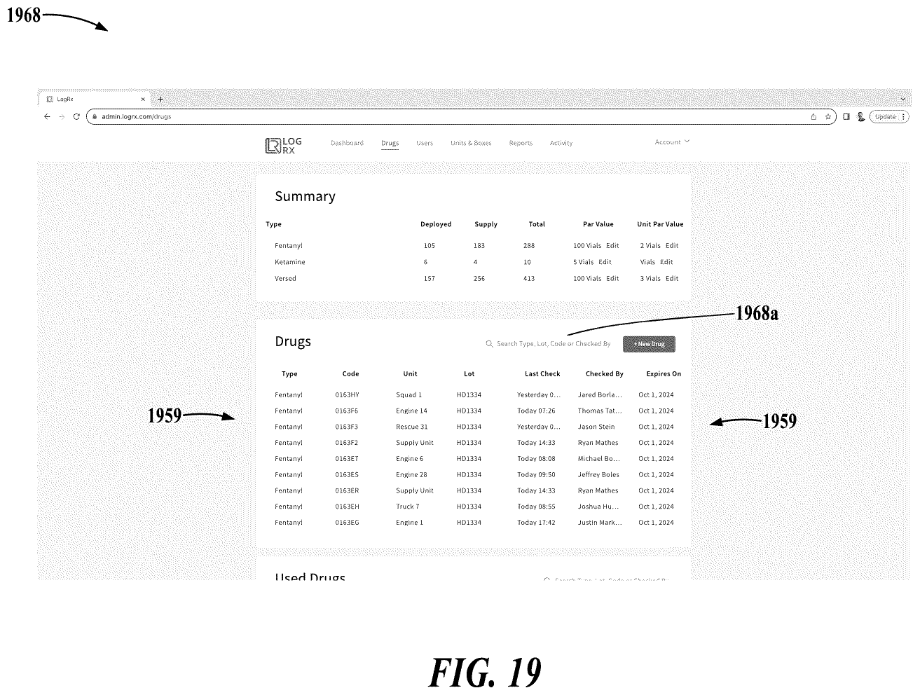

Referring to FIG. 19, an illustration shows a screenshot of a drug manifest function 1968 in an administrative portal of a digital platform. The manifest function 1968 can be searched by a user via a search bar 1968a, and can display any individual scanned vial included in the medication data 1959. The exemplary search function shown includes drug type/name or code, lot number, or user name (e.g. the name of a user who performed daily/inventory checks) as search criteria. In the depicted example, medication data 1959 displayed in the returned search result is associated with a particular lot of Fentanyl having the number “HD1334”. The data 1959 includes vial codes, unit or vial box location/tracking info, daily check or inventory check info, and expiration dates.

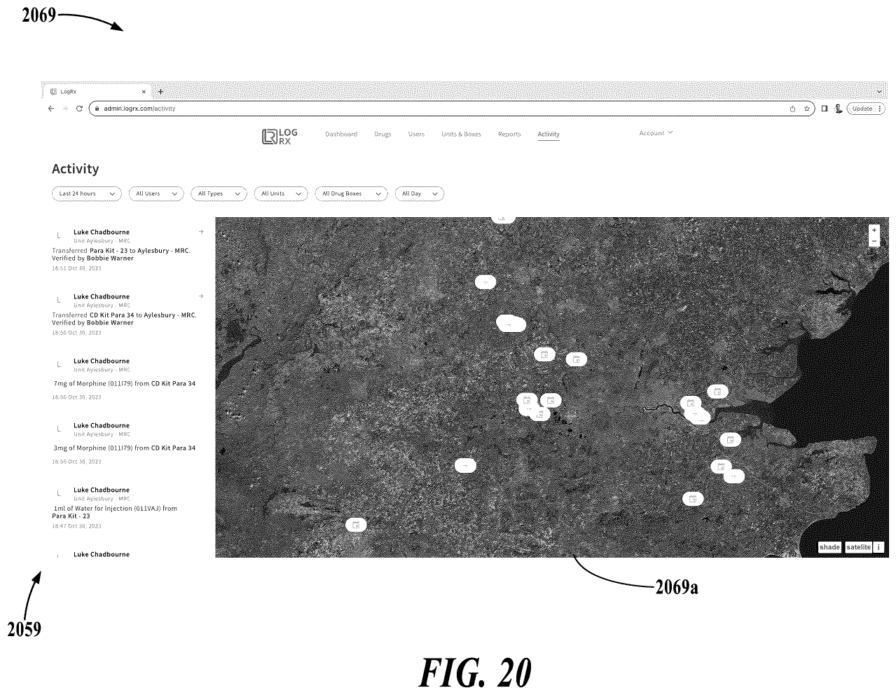

Referring to FIG. 20, an illustration shows a screenshot of a medication tracking function 2069 in an administrative portal of a digital platform. An exemplary portal's tracking function 2069 uses GPS to track the aforementioned time-stamped locations of the scanned medication vials and vial boxes after the scanning of the identifiers occurs. In a preferred embodiment, both the mobile application and administrative portal utilize GPS in their respective tracking functions. The depicted screen of the portal's tracking function 2069 includes medication data 2059 associated with a particular user, listing drug identification info as well as activities performed with the drugs by the user, along with dates and locations/units (i.e. vial box locations) for these activities. An exemplary portal's tracking function 2069 further provides an interactive GPS supported map 2069a pinned with drug location icons, these icons providing accurate visual representations of the listed drug location data. In one example, each icon and its associated list item are interactively clickable, such that clicking a list item causes a visual change (such as highlighting) in its associated map icon, and vice versa.

Many variations may be made to the embodiments described herein. All variations are intended to be included within the scope of this disclosure. The description of the embodiments herein can be practiced in many ways. Any terminology used herein should not be construed as restricting the features or aspects of the disclosed subject matter. The scope should instead be construed in accordance with the appended claims.

There may be many other ways to implement the disclosed embodiments. Various functions and elements described herein may be partitioned differently from those shown without departing from the scope of the disclosed embodiments. Various modifications to these implementations may be readily apparent to those skilled in the art, and generic principles defined herein may be applied to other implementations. Thus, many changes and modifications may be made to the disclosed embodiments, by one having ordinary skill in the art, without departing from the scope of the disclosed embodiments. For instance, different numbers of a given element or module may be employed, a different type or types of a given element or module may be employed, a given element or module may be added, or a given element or module may be omitted.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

1. A mobile system for tracking and managing government-regulated medication and associated activities comprising:
(a.) portable medication securing units, the units further comprising:
i. medication vials, each vial having a cap portion and body portion, the vials being labeled with vial identifiers;
ii. vial boxes configured to store the medication vials, the boxes being labeled with box identifiers;
(b.) at least two mobile computing devices, each device having scanners configured for optical identification of the vial and box identifiers, the scanners further configured for biometric identification of a primary user or a witness, the primary user operating a first mobile device, the witness operating a second mobile device;
(c.) a digital platform having cloud-connectivity and utilizing an Application Programming Interface (API), the platform further comprising:
i. a mobile application installed on the mobile devices and operable by the primary user or witness, the application having identification, activity, tracking, inventory, and multiuser verification functions, these functions configured to process and display medication data associated with the scanned medication securing units, the primary user, and the witness, the functions further configured to record said medication data locally and to the cloud;
ii. an administrative portal operable by an administrator and having dashboard, tracking, inventory, trend-tracking, and report-generating functions, the medication data accessible and manageable via the portal functions in real-time via the cloud; and
wherein the multiuser verification function is configured to biometrically identify the witness on the second mobile device, provide a scannable witness code on the second mobile device for the primary user to scan using the first mobile device so to verify the identity of the witness, and record verification information associated with the verified witness identification event as medication data, and wherein the medication data is integrated into the digital platform via the cloud following the verified witness identification event.

2. The medication tracking and management system of claim 1, wherein each vial identifier includes a unique alpha-numeric code for tracking, the code being visible in both data matrix barcode form for scanning and printed form for manual entry, wherein the witness code is provided for a limited period of time, and wherein the locally recorded medication data is automatically uploaded to the digital platform and accessible from the portal via the API when cloud access is available.

3. The medication tracking and management system of claim 2, wherein the alpha-numeric code is six digits, the data matrix barcode form being positioned on the cap portion while the printed form is positioned on the body portion, wherein the vial identifiers are color-coded, each vial identifier being configured to fit any medication vial with a minimum width of 0.5″, and wherein each box identifier is a self-locking, tamper-evident, one-piece component with multiple locking positions, the box identifier further including a unique alpha-numeric code for tracking, the code being visible in both data matrix barcode form for scanning and printed form for manual entry.

4. The medication tracking and management system of claim 2, wherein the mobile application's identification function provides medication data that includes identification information, the identification information further including a medication name, vial code, dose information, expiration date, box code, and lot number for each successfully scanned vial, wherein the identification function is further configured to automatically scan the vial identifier or box identifier via the optical scanners when either identifier lies within an optimal viewing region, and wherein the identification function includes a scan status indicator.

5. The medication tracking and management system of claim 4, wherein the activity function is configured to allow the primary user to transfer, administer, or waste the medication after each scan, said activities being recorded as medication data that includes an incident number for each activity, wherein the mobile application's tracking function uses GPS to track time-stamped locations of the vials or boxes when they are scanned, checked, transferred, administered, or wasted, said tracking information being recorded as medication data, wherein the mobile application further comprises a daily check function that requires a physical scan of the medication vial or box, wherein the multiuser verification function includes a user identification function that is configured to verify the identity of the primary user, alternate users, or guests via login credentials or other verification information, wherein the verification information further comprises date, time, and witness information associated with the verified witness identification event, and wherein the mobile application's inventory function is configured to display the medication data, the medication data further including quantity information associated with the medication securing units.

6. The medication tracking and management system of claim 5, wherein the administrative portal's inventory function further comprises a high level function and detailed function for displaying grouped and individualized medication data respectively, wherein the portal's tracking function uses GPS to track the time-stamped locations of the vials and boxes, wherein the portal further includes a searchable manifest function configured to display any individual vial included in the medication data, wherein the portal further includes a usage data function configured to monitor and manage the medication activity data, including administered vs. wasted statistics associated with the scanned vials, wherein the trend-tracking function is configured to track the time of every daily check and visually provide trends and outliers in the associated medication data, and wherein the report-generating function is configured to provide one-touch comprehensive report generation associated with the medication data.

7. A method utilizing a system for tracking and managing medication and associated activities, the method comprising:
(a.) Logging in a primary user to a digital platform's mobile application on a first mobile computing device;
(b.) Navigating to a drug identification function found in the mobile application;
(c.) Scanning a medication vial or vial box via a vial identifier or box identifier, respectively;
(d.) Selecting an activity for the scanned item via an activity function found in the mobile application;
(e.) Verifying the activity via a multiuser verification function found in the mobile application and using a witness by selecting biometric identification;
(f.) Logging in the witness to the mobile application on a second mobile computing device;
(g.) Providing biometric identification of the witness on the second mobile computing device;
(h.) Generating a witness code on the second mobile computing device;
(i.) Scanning the witness code on the first mobile computing device to complete verification;
(j.) If the first mobile computing device is offline, then saving medication identification data, medication activity data, and witness verification data locally;
(k.) If the first mobile computing device is online, then saving the above data to the digital platform via the cloud;
(l.) Viewing the data on the first mobile computing device; and,
(m.) Logging in an administrator to the digital platform's administrative portal on a third computing device for monitoring and management of cumulative medication, user, and witness data.

8. The method of claim 7, wherein the activities in Step (d.) include administering, transferring, or wasting the scanned item.

9. The method of claim 8, wherein verifying the activity in Step (e.) includes an option for self-verifying the primary user, wherein the witness can be an alternate user or a guest, wherein if selecting self or alternate user, then completing verification following successful identification of the primary user or alternate user, the alternate user entering login credentials on the first mobile computing device, and wherein if selecting guest, then providing guest information on the first mobile computing device.

10. The method of claim 9, wherein the data-saving to the cloud in Step (k.) occurs automatically once the first mobile computing device gains cloud access.

11. The method of claim 10, wherein the data-viewing on the first mobile computing device in Step (l.) includes accessing tracking and inventory functions found in the mobile application.

12. The method of claim 11, wherein the monitoring and management of the cumulative data on the administrative portal in Step (m.) includes accessing dashboard, tracking, inventory, trend-tracking, and report-generating functions found in the portal.

13. A system for mobile substance tracking, the system comprising:
(a.) substance securing units;
(b.) at least one mobile computing device, the device(s) having scanners configured for identification of the substance securing units, the scanners further configured for biometric identification of a primary user or a witness; and,
(c.) a digital platform having cloud-connectivity, the platform further comprising:
i. a mobile application installed on the mobile device(s) and operable by the primary user or witness, the application configured to process, display, and locally record substance data associated with the scanned substance securing units, the primary user, and the witness, the mobile application including a multiuser verification function configured to biometrically identify and verify the witness via the scanners, wherein the substance data is uploadable into the digital platform via the cloud;
ii. an administrative portal operable by an administrator and configured to cumulatively monitor and manage the substance data recorded by the mobile application in real-time via the cloud.

14. The mobile substance tracking system of claim 13, wherein the substance securing units comprise substance vials stored in vial boxes, the vials having vial identifiers, the boxes having box identifiers, both types of identifiers being scannable.

15. The mobile substance tracking system of claim 14, wherein the multiuser verification function is configured to biometrically identify the witness on a second mobile device, provide a scannable witness code on the second mobile device for the primary user to scan using a first mobile device so to verify the identity of the witness, and record verification information associated with the verified witness identification event as substance data, and wherein the witness code is provided for a limited period of time.

16. The mobile substance tracking system of claim 15, wherein the mobile application further includes identification, activity, tracking, and inventory functions for processing, displaying, and recording the substance data, and wherein the locally recorded substance data is automatically uploaded to the digital platform and accessible from the portal via an Application Programming Interface (API) when cloud access is available.

17. The mobile substance tracking system of claim 16, wherein the administrative portal includes dashboard, tracking, inventory, trend-tracking, and report-generating functions for monitoring and managing the substance data.

18. The mobile substance tracking system of claim 17, wherein the identification function provides substance data that includes identification information scanned from the vials and boxes, wherein the activity function is configured to allow the primary user to transfer, administer, or waste a substance after each scan, wherein the mobile application's tracking function uses GPS to track time-stamped locations of the vials or boxes when they are scanned, checked, transferred, administered, or wasted, wherein the mobile application's inventory function is configured to display the substance data, the substance data further including quantity information associated with the substance securing units, and wherein the multiuser verification function includes a user identification function that is configured to verify the identity of the primary user, alternate users, or guests via login credentials or other verification information.

19. The mobile substance tracking system of claim 18, wherein the administrative portal further includes a manifest function configured to search for and display any individual substance vial in the inventory, and a usage data function configured to monitor and manage substance data associated with the actions taken with the activity function, wherein the administrative portal's inventory function further comprises a high level function and detailed function for displaying grouped and individualized substance data respectively, and wherein the portal's tracking function uses GPS to track the time-stamped locations of the vials and boxes.

20. The mobile substance tracking system of claim 19, wherein each vial has a cap portion and body portion, wherein each vial identifier includes a unique alpha-numeric code for tracking, the code being visible in both data matrix barcode form for scanning and printed form for manual entry, the data matrix barcode form being positioned on the cap portion while the printed form is positioned on the body portion, and wherein each box identifier is a self-locking, tamper-evident, one-piece component with multiple locking positions, the box identifier further including a unique alpha-numeric code for tracking, the code being visible in both data matrix barcode form for scanning and printed form for manual entry.
